(19) United States
(12) Patent Application Publication
(10) Pub. No.: US 2013/0331326 A1
(43) Pub. Date: Dec. 12, 2013

(54) METHOD OF INDUCING HEMATOPOIETIC RECONSTRUCTION

(71) Applicant: John P. Chute, Durham, NC (US)

(72) Inventor: John P. Chute, Durham, NC (US)

(73) Assignee: DUKE UNIVERSITY, Durham, NC (US)

(21) Appl. No.: 14/000,679

(22) PCT Filed: Feb. 21, 2012
(86) PCT No.: PCT/US12/25894

§ 371 Date: Aug. 21, 2013

Related U.S. Application Data
(60) Provisional application No. 61/444,893, filed on Feb. 21, 2011.

(51) Int. Cl. A61K 38/18

(52) U.S. Cl. CPC A61K 38/1808
USPC 514/9.6; 530/300; 435/375

(57) ABSTRACT

The present invention relates, in general, to hematopoietic reconstruction and, in particular, to a method of inducing hematopoietic reconstruction using EGF. The invention also relates to compounds and compositions suitable for use in such a method.

This application claims priority from U.S. Provisional Application No. 61/444,893, filed Feb. 21, 2011, the entire content of which is incorporated herein by reference.

This invention was made with government support under Grant Number HL-086998-01 awarded by the National Institutes of Health. The government has certain rights in the invention.

### TECHNICAL FIELD

The present invention relates, in general, to hematopoietic reconstruction and, in particular, to a method of inducing hematopoietic reconstruction in a subject using epidermal growth factor (EGF). The invention also relates to compounds and compositions suitable for use in such a method.

### BACKGROUND

EGF is FDA approved for use in the treatment of epithelial wounds (e.g., skin wounds). EGF receptor (EGFR) signaling is known to be important in regulating epithelial tumors, such as breast cancer, and erlotinib and other EGFR inhibitors have been developed for the treatment of such tumors. EGF signaling has not been previously shown to have any role in regulating hematopoietic stem cells (HSCs), mature hematopoietic cells or hematologic malignancies.

HSCs can be found in proximity to bone marrow (BM) sinusoidal vessels (Kiel et al, Cell 121:1109-1121 (2005)) and recent studies have implicated endothelial cells (ECs) in regulating both HSC homeostasis and regeneration (Chute et al, Blood 105:576-583 (2005), Himburg et al, Nat. Med. 16:475-482 (2010), Salter et al, Blood 113:2104-2107 (2009), Butler et al, Cell Stem Cell 6:251-264 (2010), Hooper et al, Cell Stem Cell 4:263-274 (2009), Montfort et al, Experimental Hematology 30:950-956 (2002), Li et al, Stem Cell Res. 4:17-24 (2010), Ding et al, Nature 481:457-462 (2012)). Ding et al (Nature 481:457-462 (2012)) reported that maintenance of the HSC pool in mice was dependent upon the expression of stem cell factor (SCF) by BM ECs or perivascular cells, demonstrating the important role of ECs and perivascular cells in maintaining the HSC pool during homeostasis.

It has been shown that adult sources of human ECs produce soluble growth factors which promote the expansion of murine and human HSCs in vitro (Chute et al, Blood 105:576-583 (2005)) and support the regeneration of murine and human HSCs in vitro following radiation exposure (Chute et al, Blood 105:576-583 (2005), Himburg et al, Nat. Med. 16:475-482 (2010), Chute et al, Blood 100:4433-4439 (2002), Chute et al, Exp. Hematol. 32:308-317 (2004), Muramoto et al, Biol. Blood Marrow Transplant 12:530-540 (2006)). It has also been demonstrated that systemic infusion of autologous or allogeneic ECs accelerates BM HSC reconstitution and hematologic recovery in vivo following radiation-induced myelosuppression (Salter et al, Blood 113:2104-2107 (2009), Chute et al, Blood 109:2365-2372 (2007)). Hooper et al (Cell Stem, Cell 4:263-274 (2009)) also demonstrated a requirement for VEGFR2⁺ sinusoidal ECs to allow for hematologic recovery following total body irradiation. Similarly, systemic delivery of anti-VEcadherin (Salter et al, Blood 113:2104-2107 (2009), Butler et al, Cell Stem Cell 6:251-264 (2010)), which inhibits BM vasculogenesis, significantly delays hematologic recovery following myelosuppression (Salter et al, Blood 113:2104-2107 (2009), Butler et al, Cell Stem Cell 6:251-264 (2010)). However, the precise mechanisms through which BM ECs regulate hematopoietic regeneration remain unknown.

The present invention results, at least in part, from the discovery that EGF regulates hematopoiesis and hematopoietic reconstitution

### SUMMARY OF THE INVENTION

In general, the present invention relates to hematopoietic reconstruction. More specifically, the invention relates to a method of inducing hematopoietic reconstruction in a subject using EGF. The invention also relates to compounds and compositions suitable for use in such a method.

Objects and advantages of the present invention will be clear from the description that follows.

### BRIEF DESCRIPTION OF THE DRAWINGS

FIGS. 1A and 1B. Treatment with EGF induces regeneration of BM stern and progenitor cells. FIG. 1A CFCs. FIG. 1B. CFU-S12.

FIGS. 2A-2D. Systemic administration of EGF induces hematopoietic reconstitution in vivo.

FIGS. 3A-3K. Tie2⁺ BM ECs produce EGF and EGF mediates HSC regeneration following irradiation. (FIG. 3A) Non-contact culture of 300 cGy-irradiated BM KSL cells with primary BM ECs from Tie2Cre;Bak1^{-/-};Bax^{FL/-} mice (FL/−) supported significantly increased recovery of total cells (left), CFCs (middle) and CFU-S12 (right) compared to culture with BM ECs from Tie2Cre;Bak1^{-/-};Bax^{Fl/+} mice (FL/+) or cytokines alone (Thrombopoietin, SCF, Flt-3 ligand, TSF). *P-0.003 and ̂P=0.04 versus TSF and FL/+, respectively, for total cells (means±SEM, n=3-7/condition); *P<0.0001 and ̂P<0.0001 versus TSF and FL/+, respectively, for CFCs (means±SEM, n=3/condition, Student's 2-tailed t-test); *P=0.04 and ̂P=0.02 versus TSF and FL/+, respectively, for CFU-S12 (means±SEM, n=3-5/condition). (FIG. 3B) BM serum was collected from FL/− and FL/+ mice prior to irradiation and at 7 days post-750 cGy TBI. BM KSL cells were irradiated with 300 cGy in vitro and placed in culture with TSF alone, TSF+serum from FL/− mice or FL/+ mice. At day +7 of culture, cells plated with BM serum from non-irradiated or irradiated FL/− mice contained significantly increased total cells (left, *P=0.02 and *P=0.01, means±SEM, n=2-3/group). CFC recovery was also significantly increased in the cultures containing serum from irradiated FL/− mice compared to cultures with FL/+ serum (right, *P<0.0001, means±SEM, n=3/group, Student's 2-tailed t-test). (FIG. 3C) The bar graph shows the fold changes in mean concentrations of the identified cytokines in BM serum from Tie2Cre;Bak1^{-/-};Bax^{FL/-} mice compared to Tie2Cre;Bak1^{-/-};Bax^{FL/+} mice at 6 hours following 750 cGy TBI. Cytokines that were increased in the BM of Tie2Cre;Bak1^{-/-};Bax^{FL/-} mice (left) and decreased (right) compared to Tie2Cre;Bak1^{-/-};Bax^{FL/+} mice are shown. Analysis of cytokine concentrations was performed using the Quantibody mouse cytokine array (n=3 mice per condition). (FIG. 3D) Tie2Cre;Bak1^{-/-};Bax^{FL/-} mice (red line) have higher concentrations of EGF in the BM serum in non-irradiated state, at 6 hrs post-750 cGy and at 7 days post-750 cGy compared to C57B16 mice (black line) and Tie2Cre;Bak1^{-/-};Bax^{FL/+} mice (blue line). *P=0.02, **P=0.04, ***P=0.04 vs Tie2Cre;Bak1^{-/-};Bax^{FL/+} mice, n=3/condition, means±SEM, by Student's 1-tailed t-test. (FIG. 3E) FL/− ECs have significantly increased expression of EGF compared to C57B16 (B16) ECs and FL/+ECs by qRT-PCR. *P=0.002 and ̂P=0.003 vs B16 ECs and FL/+ ECs, means±SEM (n=3/group, left, Student's 2-tailed t-test). (FIG. 3F) EGFR expression is enriched in BM 34⁻ KSL HSCs from C57B16 mice, *P=0.02, *P=0.008, and *P=0.04 for difference between lin⁺, lin⁻, and 34⁻KSL cells, respectively, compared to whole BM (WBM, means±SEM, n=3-5/group). (FIG. 3G) BM KSL cells were exposed to 300 cGy and then cultured with TSF or TSF+20 ng/mL EGF and levels of phosphorylated EGFR-Y845 are shown (15 minutes). *P=0.008 (means±SEM, n=3/group, right, Student's 2-tailed t-test). (FIG. 3H) FL/− mice have increased BM MECA⁺ vasculature compared to C57B16 (B16) and FL/+ mice. Representative BM sections stained with MECA (brown) and hematoxylin (left). Scale bar is 50 microns. Quantitation of BM MECA⁺ cells normalized by surface area (right). *P=0.02 and ̂P=0.02 vs B16 and FL/+, means±SEM (n=3-14/group). (FIG. 3I) EGF mediates BM progenitor cell regeneration following radiation injury. Bar graphs show the CFC and CFU-S 12 content at day 7 following culture of 300 cGy-irradiated BM KSL cells with FL/+ ECs with and without 20 ng/mL EGF; *P=0.01 and *P=0.0003, respectively, for CFCs and CFU-S12 versus FL/+ ECs (top left). CFC and CFU-S12 content of day 7 cultures of irradiated BM KSL cells with FL/− ECs with either anti-EGF antibody (1 μg/mL) or isotype antibody; *P=0.001 for CFCs (n=8-9/group) and *P=0.01 for CFU-S12 (n=8/group)(top right). CFC and CFU-S12 content of day 7 cultures of irradiated BM KSL cells with TSF alone and TSF with 20 ng/mL EGF; *P=0.0002 and *P=0.0003, respectively, for CFCs (n=8/group) and CFU-S 12 (n=9-12/group) versus TSF alone; means±SEM (bottom). (FIG. 3J) Bar graphs show PB donor CD45.1⁺ cell engraftment at 8 weeks post-transplant in recipient CD45.2⁺ mice following competitive transplantation of the progeny of 1×10³ BM 34⁻KSL cells that were irradiated with 300 cGy and cultured with either TSF alone or TSF+EGF for 7 days. Mean levels of engraftment of total CD45.1⁺ cells (n=7-9/group), myeloid cells (Mac-1/Gr-1), B cells (B220) and T cells (Thy1.2) are shown (top). *P=0.002 for CD45.1⁺ cell engraftment and *P=0.002 for myeloid cell engraftment. Total PB CD45.1⁺ cell engraftment at 4, 8, and 12 weeks post-transplant is also shown (bottom, red lineTSF+EGF culture, black line=TSF alone). *P=0.002 at 8 weeks post-transplant (means±SEM). (FIG. 3K) EGF mediates the expansion of non-irradiated BM HSCs. BM KSL from C57B16 mice were cultured with TSF with and without 20 ng/ml EGF for 7 days. KSL cells and CFU-S12 were increased significantly in cultures supplemented with EGF. *P=0.03 and *P=0.004, (means±SEM, n=4-6/group). Mice that were competitively transplanted with the progeny of 100 BM 34⁻KSL cells cultured with TSF+EGF cultures had significantly higher donor CD45.1⁺ cell repopulation compared to recipients of progeny from TSF cultures alone. *P=0.04 (at right, means±SEM, n=4-5 mice/group, Student's 2-tailed t test). The Mann-Whitney test was applied for all statistical analyses unless otherwise noted.

FIGS. 4A-4J. Systemic administration of EGF accelerates HSC and progenitor cell regeneration in vivo. BM hematopoietic content was measured at day +7 following 700 cGy TBI and daily intraperitoneal (IP) administration of 0.5 μg/G EGF or saline×7 days. (FIG. 4A) Schematic diagram of treatment of mice following TBI with either EGF or normal saline (NS) IP×7 days and subsequent collection of BM at day +7 for both progenitor cell assays and competitive transplantation into congenic, lethally irradiated mice to measure HSC repopulating capacity. (FIG. 4B) EGF-treated mice displayed preserved BM cellularity, whereas saline-treated mice were hypocellular (hematoxylin and eosin stain, scale bar 250 microns). BM cell counts were significantly increased in EGF-treated mice. *P=0.003 (means±SEM, n=6/group). (FIG. 4C) Representative flow cytometric analysis of BM c-kit⁺sca-1⁺ cells within the lin⁻ gate (KSL) from non-irradiated mice or mice exposed to 700 cGy and treated with EGF or saline for 7 days. EGF-treated mice displayed significantly increased BM KSL cells compared to the saline-treatment group. *P=0.008 (means±SEM, n=6/group). (FIG. 4D) BM CFCs and CFU-S12 at day +7 in saline-treated vs. EGF-treated, irradiated mice. *P<0.0001 and *P=0.03 for CFCs and CFU-S12 (means±SEM, n=3-5/group). (FIG. 4E) The mean PB engraftment of donor CD45.2⁺ cells in recipient CD45.1⁺ mice is shown at 12 weeks following competitive transplantation of BM cells harvested from C57B16 mice which were irradiated with 700 cGy TBI and treated with either EGF or saline×7 days. *P=0.02 for CD45.2⁺ cell engraftment (n=3-8/group, Mann-Whitney test). Myeloid (Mac-1), B cell (B220) and T cell (Thy 1.2) engraftment levels are shown at 12 weeks. *P=0.01 for Myeloid and *P=0.02 for B cell engraftment. (FIG. 4F) Donor CD45.2⁺ cell engraftment is shown at 12 weeks in secondary transplant recipient mice (CD45.1⁺) following (non-competitive) transplantation of BM cells collected from primary mice that had been transplanted with BM cells collected at day +14 from irradiated, EGF-treated donor mice or irradiated, saline-treated donor mice. The entire BM cell contents of both femurs from each primary recipient mouse were transplanted into individual secondary recipient mice. Mean CD45.2⁺ cell, myeloid, B220 and Thy 1.2 cell engraftment was significantly increased in the EGF-treatment group compared to the saline-treatment group. *P=0.01 for CD45.2⁺ engraftment, *P=0.02, *P=0.02 and *P=0.02 for myeloid, B cell and T cell engraftment, respectively. n=3-8/group, means±SEM, 2-tailed t test. (FIG. 4G) C57B16 mice were exposed to 700 cGy TBI and subsequently treated with 10 μg/G erlotinib or water via oral gavage starting 2 hours post-TBI and daily through day +14. Schematic diagram of TBI and treatment of C57B16 mice, with evaluation of BM progenitor cell content and HSC repopulating capacity via competitive transplantation assay at day +14. (FIG. 4H) BM cellularity is shown from irradiated mice treated with erlotinib or water at day +14 post-TBI (hematoxylin and eosin stain, scale bar 250 microns). (FIG. 4I) BM CFCs (per 2×10⁴ cells) and BM CFU-S12 were significantly reduced in erlotinib-treated mice at day +14 compared to control mice. *P=0.008 and *P=0.04 (means±SEM, n=3/group). (FIG. 4J) Mean levels of donor CD45.2⁺ cell engraftment are shown in the PB of recipient CD45.1⁺ mice at 12 weeks following competitive transplantation of 5×10⁵ BM cells harvested at day +14 from irradiated, erlotinib-treated mice or irradiated, water-treated controls (n=3-4/group, means±SEM). *P=0.007 for total CD45.2⁺ cell engraftment. *P=0.04 and *P-0.003 for myeloid cell and T cell differences, respectively. PB donor cell engraftment over time in recipient mice described above; red line=recipients of BM cells from erlotinib-treated donor mice, black line=recipients of BM cells from water-treated mice. *P=0.002, *P=0.0002, and *P=0.007 at 4, 8, and 12 weeks, respectively (n=3-4/group). Student's 2-tailed t-test was applied for statistical analysis unless otherwise noted.

FIGS. 5A-5D. (FIG. 5A) Schematic diagram of irradiation and treatment of Tie2Cre;Bak1^{-/-};Bax^{Fl/-} mice with erlotinib or water starting 3 days prior to TBI with evaluation of BM progenitor cell content and HSC repopulating capacity via competitive transplantation at +2 hours after TBI. (FIG. 5B) Erlotinib-treated Tie2Cre;Bak1^{-/-};Bax^{Fl/-} mice demonstrated decreased BM KSL cells and decreased BM CFU-S12 content compared to water-treated Tie2Cre;Bak1−/−;Bax^{Fl/-} mice, *P=0.02 and *P=0.046 for BM KSL and CFU-S12, respectively (n=2-5, means±SEM). (FIG. 5C) The percentage of BM KSL cells with phosphorylation of EGFR-Y845 is shown for erlotinib-treated Tie2Cre;Bak1^{-/-};Bax^{Fl/-} mice and water-treated Tie2Cre;Bak1^{-/-};Bax^{Fl/-} mice (n=2, means±SEM). (FIG. 5D) Bar graphs show mean PB engraftment of donor CD45.2⁺ cells in recipient CD45.1⁺ mice at 12 weeks following transplant of 3×10⁵ BM cells harvested from Tie2Cre;Bak1^{-/-};Bax^{Fl/-} mice at +2 hrs following 300 cGy TBI and subsequent to pre-treatment with erlotinib or water. 1×10⁵ host CD45.1⁺ BM cells were administered as competitor cells. *P=0.03 and *P=0.02 for CD45.2⁺ cell and T-cell engraftment (means±SEM, n=4-6/group, top). Donor CD45.2⁺ cell engraftment is shown over time in recipient mice transplanted with BM cells harvested from Tie2Cre;Bak1^{-/-};Bax^{Fl/-} mice that were pre-treated either erlotinib (red line) or water (black line) and then irradiated with 300 cGy TBI. Mice transplanted with BM cells from erlotinib-treated donor cells (red line) demonstrated significantly lower engraftment at 4, 8 and 12 weeks post-transplant compared to mice transplanted with BM cells from water-treated donors (black line). *P=0.009, *P<0.0001 and *P=0.03 for differences at 4 weeks, 8 weeks and 12 weeks, respectively (bottom). A Student's 2-tailed t-test was used for all statistical analyses.

FIGS. 6A-6G. Deletion of EGFR inhibits hematopoietic progenitor cell regeneration. (FIG. 6A) RTPCR for EGFR expression was measured in BM lin⁻ cells from VavCre;EGFR^{FL/FL} (FL/FL) mice versus VavCre;EGFR^{+/+}(+/+) control mice. *P=0.008 (means±SEM, n=5/group) (FIG. 6B) The baseline yield of total cells and CFCs at 72 hours of culture of BM lin⁻ cells from EGFR FL/FL mice versus EGFR+/+ mice is shown. *P=0.03 and *P=0.002 for total cells and CFCs, respectively (means±SEM, n=5-6/group). (FIG. 6C) Erlotinib-treatment of EGFR+/+ BM lin⁻ cells caused a decreased yield in total cells and CFCs in culture compared to EGFR FL/FL BM lin⁻ cells. *P=0.004 and *P=0.04 for total cells and CFCs, respectively (n=8/group). (FIG. 6D) EGFR expression in BM lin⁻ cells from VavCre;EGFR^{Fl/+} (FL/+) mice compared to BM lin⁻ cells from EGFR+/+ mice. *P=0.008 (means±SEM, n=5/group. (FIG. 6E) No differences were observed in complete blood counts (n=10-15/group) or (FIG. 6F) BM CFCs between EGFR+/+ mice versus EGFR FL/+ mice (n=6/group). (FIG. 6G) EGFR FL/+ mice contained decreased BM CFCs and BM SLAM⁺KSL cell content compared to EGFR+/+ mice at day +7 following 500 cGy TBI. *P=0.002 (n=6/group) and *P=0.004 (n=6-11/group) for CFCs and SLAM⁺KSL cells, respectively. The Mann-Whitney test was applied for all statistical analyses.

FIGS. 7A-7G. EGF promotes HSC survival and proliferation early following radiation injury. (FIG. 7A) Mean percentage of Annexin⁺ BM KSL cells (from C57B16 mice) at 72 hours of culture with TSF or TSF plus 20 ng/ml EGF after 300 cGy is shown. *P=0.002 (means±SEM n=8/group, left). Mean percentage of Annexin⁺ CD45⁺MECA⁻ cells in the BM of adult C57B16 mice at day +7 following 700 cGy TBI and treatment with either saline or EGF is shown. *P=0.03 (means±SEM, n=4-5/group, right). (FIG. 7B) Mean percentage of Annexin⁺ Tie2Cre;Bak1^{-/-};Bax^{Fl/-}. KSL cells at 72 hours following 300 cGy and treatment with either TSF or TSF+20 ng/ml EGF. *P=0.009 (means±SEM, n=5-6/group, left). Tie2Cre;Bak1^{-/-};Bax^{Fl/-} KSL cells were treated with TSF or TSF plus 20 ng/ml EGF in culture for 72 hours following 300 cGy. EGF-treated cells displayed increased CFCs following irradiation compared to TSF treatment. *P=0.002 (means±SEM, n=6/group, right). (FIG. 7C) At 72 hours following 300 cGy irradiation, BM KSL cells treated with TSF+EGF contained a decreased percentage of cells in G₀ and increased percentage in G₂/S/M compared to TSF-treated cells. Non-irradiated BM KSL cells reside predominantly in the G₀ (white). G₁ light gray, G₂/S/M=dark gray. *P=0.002 vs TSF for G₀ and ̂P=0.002 vs TSF for G₂/S/M, respectively (means±SEM, n=3-5/group). (FIG. 7D) A representative flow cytometric analysis is shown of BrdU incorporation in BM KSL cells in adult C57B16 mice at day 7 following 700 cGy TBI and treatment with EGF or saline (left); mean BrdU incorporation was significantly increased in BM KSL cells in EGF-treated mice. *P=0.02 (at right, means±SEM, n=3/group, Student's 2-tailed t-test). (FIG. 7E) Following 300 cGy, BM KSL from Tie2Cre;Bak1^{-/-};Bax^{Fl/-} were placed in culture with TSF or TSF plus 20 ng/ml EGF for 72 hours, and BM KSL cell cycle status was measured. The majority of non-irradiated cells were in G₀ (white). Following 300 cGy, the addition of EGF significantly decreased G₀ cells and increased G₂/S/M cells (dark gray) compared to TSF. *P=0.002 for G₀, TSF vs EGF (n=6/group), ̂P=0.002 for G₂/S/M, TSF vs EGF (n=6/group; G₁=light gray). (FIG. 7F) EGF mediates BM stem/progenitor cell regeneration through activation of Akt. Following 300 cGy, EGF treatment of BM KSL cells significantly increased % phospho-AKT compared to irradiated BM KSL cells treated with TSF alone (15 minutes). *P=0.03 TSF vs EGF (means±SEM, n=7-8/group). When 20 μM Ly294002 (Ly29) was added to EGF-treated BM KSL cells, % phospho-AKT significantly decreased (at left). ̂P=0.0006 EGF vs EGF+Ly29 (means±SEM, n=7-8/group). Treatment of irradiated BM KSL cells×72 hours with TSF+EGF caused a significant increase in CFCs compared to TSF alone; the addition of Ly29 blocked BM CFC regeneration in response to EGF (middle). *P<0.0001, TSF vs EGF; ̂P<0.0001 EGF vs EGF+Ly29 (means±SEM, n=9/group). Treatment with Ly 29 significantly inhibited the entry of HSCs into cell cycle in response to EGF (right). *P<0.0001 for G₀, EGF vs. TSF; ̂P=0.004 for G₂/S/M, EGF vs. TSF; *P<0.0001 for G₀, EGF+Ly vs. EGF; ̂P=0.0001 for G₂/S/M, EGF+Ly vs. EGF; n=4-5/group, means±SEM, 2-tailed t test. (FIG. 7G) EGF treatment increases phosphorylation of DNA-PK following irradiation. Representative images of C57B16 BM lin⁻ cells that were exposed to 300 cGy and then treated with TSF versus TSF+20 ng/ml EGF. At 15 minutes following treatment, BM lin⁻ cells displayed increased phospho-DNA-PK (green) staining by immunofluorescence (left, scale bar 10 microns). At 1 hour post-treatment with TSF+EGF, increased phospho-DNA-PK was quantified by FACS analysis of irradiated BM lin⁻ cells compared to BM lin⁻ cells treated with TSF alone. *P=0.04, means±SEM, n=3/group, Student's 2-tailed t test. The Mann-Whitney test was utilized for all comparisons unless otherwise noted.

FIG. 8 Pharmacologic modulation of EGFR signaling alters survival following TBI. C57B16 mice were irradiated with 700 cGy TBI and then given 0.5 μg/gram EGF (red curve) or saline intravenously (blue curve) beginning at +2 hours and then daily through day +7. Fourteen of 15 mice treated with EGF (93%) remained alive and well through day +30. Conversely, only 8 of 14 saline-treated mice (57%) survived through day +30. *P=0.02 for EGF vs. saline survival. An additional group of age matched C57B16 mice were irradiated with 700 cGy TBI and treated with 10 μg/G erlotinib (green curve) or water (black curve) via gavage beginning 3 days prior to TBI and continuing until day +14. One hundred percent of the erlotinib-treated mice (15 of 15) died by day +27 compared to 53% survival (8 of 15) through day +30 in the mice treated with water gavage. *P=0.003 for erlotinib vs. water. Log rank test was utilized for comparisons.

### DETAILED DESCRIPTION OF THE INVENTION

Hematologic toxicity continues to significantly limit the delivery of curative chemotherapy and radiotherapy in the treatment of patients with cancer. Currently, there are no available cytokines or growth factors which can be administered to accelerate reconstitution of the hematopoietic system in patients in need thereof. The studies described in the Examples that follows demonstrate that systemic administration of EGF causes a significant and pronounced acceleration in recovery of the hematopoietic stem and progenitor cell compartment in vitro and in vivo in animals following high dose total body irradiation. These results provide the foundation for the translation of EGF as a novel growth factor for patients undergoing chemotherapy or radiation therapy for cancer, as well as patients undergoing bone marrow transplantation.

The present invention relates to a method of inducing or accelerating hematologic recovery in a subject (e.g., a human or non-human mammal) in need thereof. Examples of such subjects include patients who have undergone (or who are undergoing) myelotoxic therapies, such as radiation and/or chemotherapy. The present method also has applicability, for example, in patients undergoing bone marrow transplantation (e.g., stem cell transplantation).

In accordance with the invention, EGF can be administered using any mode that results in the desired induction or acceleration of hematologic recovery. Systemic administration (e.g., via intraperitoneal injection) is preferred. The optimum amount to be administered and dosing regimen can vary, for example, with the patient, and can be determined by one skilled in the art.

The EGF can be formulated with a pharmaceutically acceptable carrier to form a composition (e.g., a sterile composition) suitable for administration. Pharmaceutically acceptable carriers are well known to those skilled in the art, saline being an example. The choice of carrier can vary, for example, with the particular method of administration.

The EGF used in the present method (e.g., human EGF) can produced, for example, recombinantly using methods well known in the art. Active fragments of LOP can also be used.

Certain aspects of the invention are described in greater detail in the non-limiting Examples that follows.

### Example 1

A cytokine array analysis was conducted of serum from the bone marrow of Bak−/−;BaxFL/− mice that had radioprotection from radiation injury. This cytokine screen revealed EGF to be highly enriched in the radioprotected mice compared to non-protected mice. In vitro studies were then performed to test recombinant EGF against murine stem cells following radiation exposure. These studies revealed that EGF induced the regeneration of stem cells after regeneration in vitro. In vivo studies were then performed in which recombinant EGF was administered via intraperitoneal injection into irradiated mice and it was found that administration of EGF systemically caused a marked acceleration in recovery of bone marrow stem and progenitor cells in irradiated, wild type mice compared to controls.

It was also found that administration of erlotinib, a specific inhibitor of EGFR signaling, caused a marked delay in hematopoietic reconstitution following total body irradiation in mice.

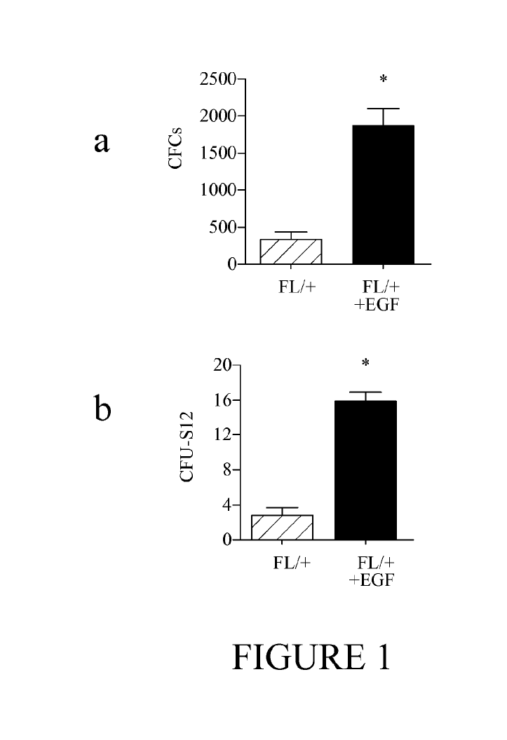

More specifically, BM ckit+sca-1+lin− (KSL) cells were irradiated with 300 cGy and then placed in culture×7 days with BaxFl+ endothelial cells (FL+) with and without EGF. At day 7, colony forming cell (CFC) and colony forming unit spleen (CFUS12) were measured. As shown in FIG. 1, treatment with EGF induced regeneration of BM stem and progenitor cells.

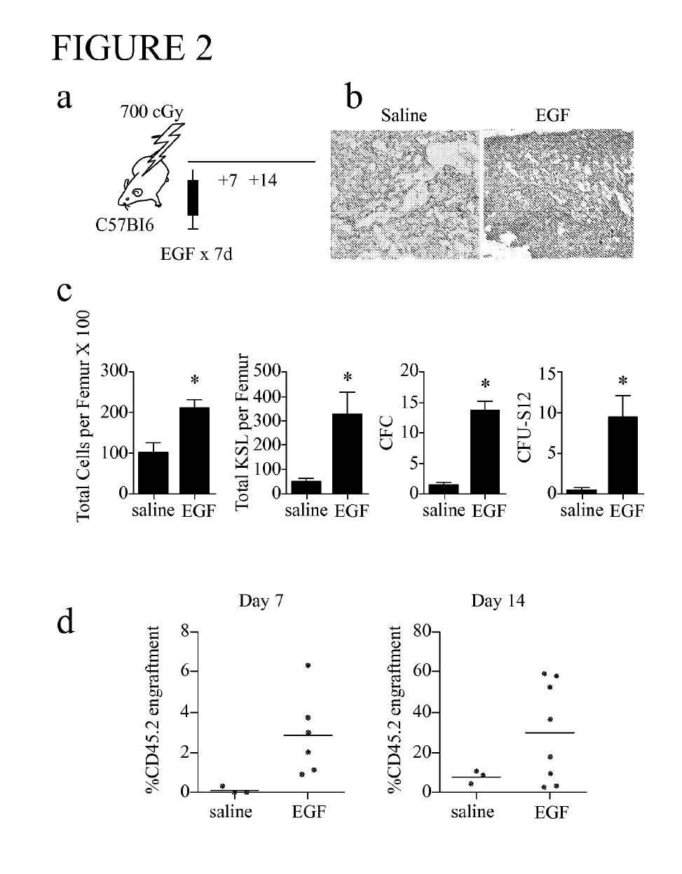

C57B16 mice were treated with 700 cGy total body irradiation (TBI) and then followed for recovery of BM hematopoietic stem and progenitor cells over time in response to EGF treatment or saline. FIG. 2A is a schematic of the experiment. FIG. 2B is a microscopic image of BM cellularity at day 7 following TBI with and without EGF treatment. In FIG. 2C, left to right, total BM cells, KSL cells, CFC and CFUS content is compared at day +7 between saline treated and EGF treated mice. In FIG. 2D, donor stem cell engraftment is shown in recipient mice transplanted with BM cells from either irradiated, saline treated or irradiated, EGF treated mice at day +7 or day +14 following 700 cGy TBI-EGF treated mice had significantly higher repopulating cell content.

In summary, EGF is overexpressed by bone marrow endothelial cells in radioprotected animals (mice). Treatment of murine HSCs with EGF induces their regeneration following exposure to high dose irradiation. Systemic administration of EGF (via intraperitoneal injection) to mice following total body irradiation causes a significant and marked acceleration in hematopoietic stern cell reconstitution and overall hematologic recovery compared to control irradiated animals. Systemic administration or erlotinib, an specific EGFR inhibitor, significantly delays hematopoietic reconstitution following total body irradiation in mice.

### Example 2

### Experimental Details

### Animals

Ten to 12 week-old C57B16 (CD45.2⁺) mice and B6.SJL (CD45.1⁺) mice were obtained from Jackson Laboratory (Bar Harbor, Me.). Tie2Cre;Bak1^{-/-}; Bax^{FL/-} and Tie2Cre;Bak1^{-/-};Bax^{FL/+} were generated as previously described (Kirsch et al, Science 327:593-596 (2010)). EGFR^{Fl/Fl} mice (Lee and Threadgill, Genesis 47:85-92 (2009)) (Mutant Mouse Regional Resource Centers, Chapel Hill, N.C.) were bred with VavCre mice (Jackson Laboratory) to generate VavCre;EGFR^{fl/+} mice. In VavCre mice, floxed alleles are excised by Cre in Vav⁺ cells and their progeny (Georgiades et al, Genesis 34:251-256 (2002), de Boer et al, Eur. J. Immunol. 33:314-325 (2003)). To generate VavCre;EGFR^{fl/fl} mice, VavCre;EGFR^{fl/+} mice were mated with EGFR^{fl/fl} mice. Mice were genotyped for the cre allele through Transnetyx, Inc (Cordova, Tenn.) and loxP-EGFR alleles as previously described (Lee and Threadgill, Genesis 47:85-92 (2009)). The deletion of EGFR in BM cells was quantified using RT-PCR (Applied Biosystems, Carlsbad, Calif.). All animal studies described herein were approved by the Duke University Animal Care and Use Committee.

### Hematopoietic Progenitor Cell Assays

BM cells were collected into PBS (Cellgro, Manassas, Va.) with 10% fetal bovine serum (Hyclone, Logan, Utah) and 1% penicillin/streptomycin (GIBCO, Grand Island, N.Y.). Viable BM cells were quantified using Trypan Blue Stain (Lonza, Basel, Switzerland) to exclude apoptotic and dead cells. Cells were then incubated with anti-c-kit, anti-Sca-1, anti-lineage cocktail, anti-CD41, anti-CD48, and anti-CD150 antibodies (Biolegend and eBiosciences, San Diego, Calif.; BD, San Jose, Calif.) to measure ckit⁺sca-1⁺lin⁻ (KSL) progenitor cells or CD150′CD41⁻CD48⁻KSL (SLAM/KSL) as previously described (Kiel et al, Cell 121:1109-1121 (2005)). Colony forming cells (CFCs) and CFU-S12 assays were also performed to measure functional hematopoietic stem/progenitor cell content. For CFCs, either whole BM or cultured lineage−cells, or KSL cells were plated onto methylcellulose (StemCell Technologies, Vancouver, BC, Canada), and colonies were scored on day 14. 1×10⁵ BM or 2×10⁵ cells were collected from donor mice and injected via tail vein into recipient C57B16 mice that had been given 950 cGy TBI. At day +12 post-injection, spleens from recipient mice were harvested and stained with Bouin's fixative solution (Ricca Chemical Company, Arlington, Tex.), and colonies were counted as previously described (Till and MeRadiat. Res. 14:213-222 (1961)). Complete blood counts were performed on a HemaVet 950 (Drew Scientific, Dallas, Tex.).

Generation and Culture of Primary BM ECs from Tie2Cre;Bak1^{-/-};Bax^{Fl/-} Mice

For isolation and generation of primary BM ECs from FL/− and FL/+ mice, whole BM was collected from bilateral femurs and passed through a 70 micron filter. BM vessel fragments were then plated, rinsed with 10% FBS, washed in PBS and treated with 0.25% trypsin-EDTA. BM vessel explants were cultured on 10% gelatin-coated wells (Sigma-Aldrich) with EGM-2 Endothelial cell growth medium-2 (Lonza) as previously described (Chute et al, Blood 105:576-583 (2005), Himburg et al, Nat. Med. 16:475-482 (2010), Chute et al, Blood 100:4433-4439 (2002), Chute et al, Exp. Hematol. 32:308-317 (2004), Yoder et al, Blood 109:1801-1809 (2007)). Wells were washed daily for 7-10 days and primary cells were passaged when confluent.

BM KSL cells from adult C57B16 mice were exposed to 300 cGy in vitro and then cultured with TSF (20 ng/ml thrombopoietin, 125 ng/ml stem cell factor, and 50 ng/ml Flt-3 ligand (TSF, R&D Systems, Minneapolis, Minn.) alone or in non-contact culture with FL/− ECs or FL/+ ECs. In some experiments, cultures were supplemented with 20 ng/ml EGF or 1 μg/ml of a blocking anti-EGF (R&D Systems, Minneapolis, Minn.). After 7 days in culture, cell progeny were collected and colony-forming cell assays (CFC) and CFU-S12 assays were performed as previously described (Chute et al, Blood 105:576-583 (2005)).

### Cytokine Array and EGF/EGFR Expression Analysis

Whole BM was collected from adult, non-irradiated Tie2Cre;Bak1^{-/-};Bax^{FL/-} mice and Tie2Cre;Bak1^{-/-};Bax^{FL/+} mice and C57B16 mice and at 6 hours and 7 days following 750 cGy TBI. After centrifugation, BM supernatants were collected into IMDM and analyzed for cytokine concentrations using Quantibody mouse cytokine array 1000, according to manufacturer's guidelines (RayBiotech, Inc., Norcross, Ga.). For analysis of expression of EGFR by C57B16 whole BM, lin⁺ cells, lin⁻ cells, and 34⁻KSL cells, total RNA was isolated from each cell population and qRT-PCR was performed using target-specific primers as previously described (Dressman et al, PLoS Med. 4:el 06 (2007)). For analysis of phosphorylation of the Y845 kinase domain of EGFR, BM KSL cells were cultured for 15 minutes with TSF or TSF+20 ng/mL EGF and then stained with Alexa Fluor 647 mouse anti-phospho-EGF-Y845 receptor antibody (BD) or isotype control.

### HSC Survival and Proliferation Assays

Three thousand C57B16 KSL cells were exposed to 300 cGy, and then placed in culture with TSF alone, TSF with 20 ng/ml EGF, or TSF, EGF, and 1 uM Ly294002 (Cell Signaling Technology, Danvers, Mass.) for 72 hours. Cell apoptosis and necrosis were analyzed by flow cytometry according to manufacturer's protocols with Annexin V-FITC and 7-AAD staining (BD, San Jose, Calif.). For analysis of phosphorylation AKT-S473, BM KSL cells were cultured for 15 minutes with TSF or TSF+20 ng/mL EGF or with 20 μM Ly294002. Cells were fixed and permeabilized with Fix Buffer I and Perm Buffer III (BD), and then stained with mouse anti-phospho-AKT-S473 PE (BD) or isotype control. Cell cycle analysis was performed by flow cytometric analysis modified from previous reports (Jordan et al, Exp. Hematol. 24:1347-1355 (1996), Chute et al, Hum. Gene Ther. 11:2515-2528 (2000), Sungartz et al, Blood 119:1308-1309 (2012)). Briefly, cells were fixed and permeabilized with 0.25% Saponin (Calbiochem, La Jolla, Calif.), 2.5% paraformaldehyde, 2% FBS in 1×PBS, and then labeled with Ki67-FITC and 7-AAD (BD). BM lin⁻ cells from VavCre;EGFR^{+/+} or VavCre;EGER^{fl/fl} were cultured with TSF or TSF+10 μM erlotinib, or TSF following 300 cGy for 72 hours, and then collected for total cell counts and CFCs analysis.

Cell proliferation was measured in C57B16 mice exposed to 700 cGy TBI and administered 5-bromo-2-deoxyridine (BrdU, BD) in drinking water from the day of irradiation until day +7. BM cells were labeled with anti-cKit PE, anti-scal PE-Cy₇, anti-lineage APC, and anti-BrdU FITC. Incorporation of BrdU was analyzed by flow cytometry according to the manufacturer's staining protocol (BD).

The phosphorylation of the T2647 domain of DNA-PK was measured using immunofluorescence and flow cytometric analysis. B16 BM lin− cells were exposed to 300 cGy and then treated with TSF or TSF+20 ng/ml EGF for 15 minutes or 1 hour. Cells were separated onto a glass slide, fixed with 4% paraformaldehyde and permeabilized with 0.3% Triton-X. Cells were stained with rabbit polyclonal DNA-PK or rabbit IgG (Abeam, Cambridge, Mass.) and donkey anti-rabbit Alexafluor 488 antibody and counterstained with Hoechst 33342 (Life Technologies, Grand Island, N.Y.).

### Competitive Repopulation Assays and Survival Studies

Competitive repopulation assays were performed using donor C57B16 mice (CD45.2⁺) that had been irradiated with 700 cGy TBI and given daily intraperitoneal injections of 0.5 μg/G EGF (R&D Systems, Minneapolis, Minn.) or 200 μl PBS starting at 2 hours post-TBI on day 0 through day +7. Competitive repopulation assays were also performed using donor C57B16 mice (CD45.2⁺) that had been irradiated with 700 cGy TBI and gavaged daily with 10 μg/G erlotinib (Genentech, San Francisco, Calif.) or 150 μl water beginning on day 0 and continued through day +14. Donor BM cells were injected via tail vein into recipient B6.SJL mice (CD45.1⁺) at a dose of 5×10⁵ cells with a competing dose of host 1×10⁵ BM MNCs. Tie2Cre;Bak1^{-/-};Bax^{FL/-} mice were gavaged daily with 10 μg/G erlotinib or water starting day −3 and given 300 cGy TBI on day 0. Erlotinib administration continued until the timepoint of donor BM cell collection and analysis. Tie2Cre;Bak1^{-/-};Bax^{FL/-} BM cells were injected via tail vein into recipient CD45.1⁺ mice at a cell dose of 3×10⁵ cells with a competing dose of host 1×10⁵ CD45.1⁺ cells. Multilineage hematopoietic reconstitution was measured in the PB of recipient mice by flow cytometry at 4, 8, and 12 weeks post-transplant. For survival studies with erlotinib administration, C57B16 mice were exposed to 700 cGy and then given 10 μg/G erlotinib or water starting day −3 and continuing daily through day +14. Age matched adult C57B16 mice were also exposed to 700 cGy TBI and then given tail vein injections with 0.5 μg/G EGF or saline beginning at +2 hrs post-TBI and then daily through day +7.

### Immunohistochemical Analyses

Femurs were decalcified and embedded in OCT media (Sakura Finetek, Torrance, Calif.) as previously described on days 7 or 14 following 700 cGy TBI with daily administration of EGF or erlotinib. Ten micrometer sections were cut using they CryoJane tape system (Instrumedics Inc, Hackensack, N.J., USA). Femurs were stained with hematoxylin and eosin or anti-mouse endothelial cell antibody (MECA-32) as previously described (Salter et al, Blood 113:2104-2107 (2009)) to assess BM cellularity and the BM vasculature after irradiation. Images were obtained using an Axiovert 200 microscope (Carl Zeiss Microscopy, Thornwood, N.Y.) or a Leica SP5 confocal microscope (Leica Microsystems Inc, Buffalo Grove, Ill.). Adobe Photoshop software (version 9.0.2, Adobe Systems, San JoSe, Calif.) was used to quantify positive signal as a measure of spatial distribution in the fields (Lehr et al, J. Histochem. Cytochem. 45:1559-1565 (1997), Lehr et al, J. Histochem. Cytochem. 47:119-126 (1999)).

### Statistical Analyses

Data are shown as means±SEM. The Mann-Whitney test (two-tailed nonparametric analysis) was used for the majority of comparisons, along with the Student's t test (two-tailed or one-tailed distribution with unequal variance). Comparisons of overall survival were performed using a Log rank test.

### Results

### Tie2Cre;Bak1^{-/-};Bax^{FL/-} Mice Secrete EGF and EGF Mediates USC Regeneration In Vitro

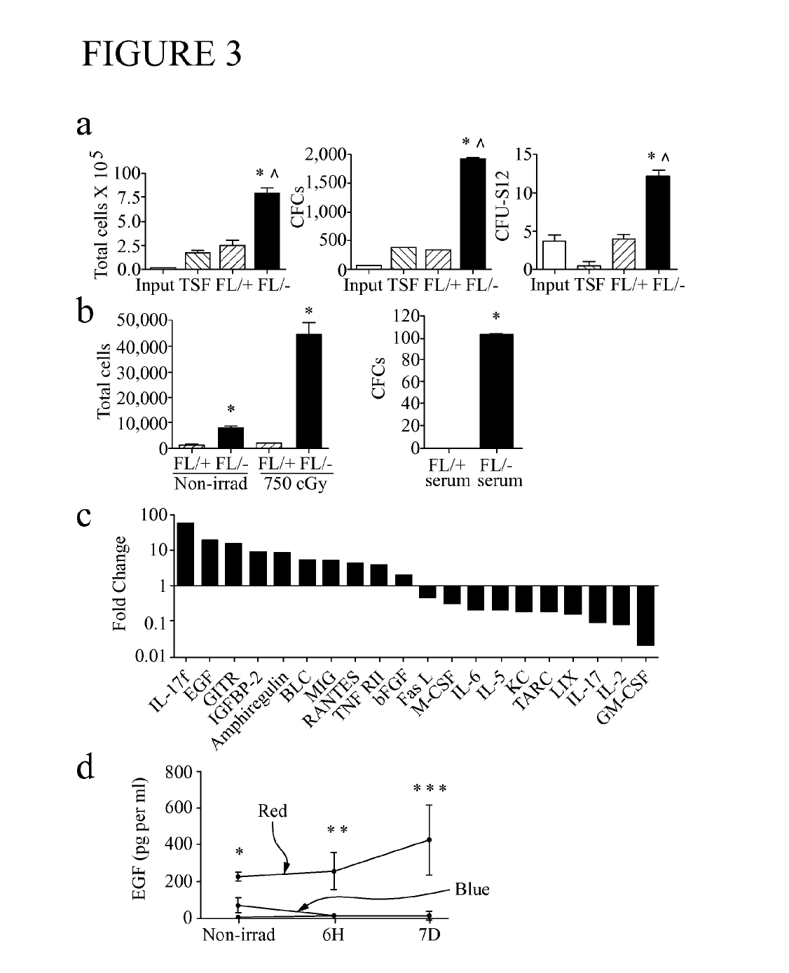

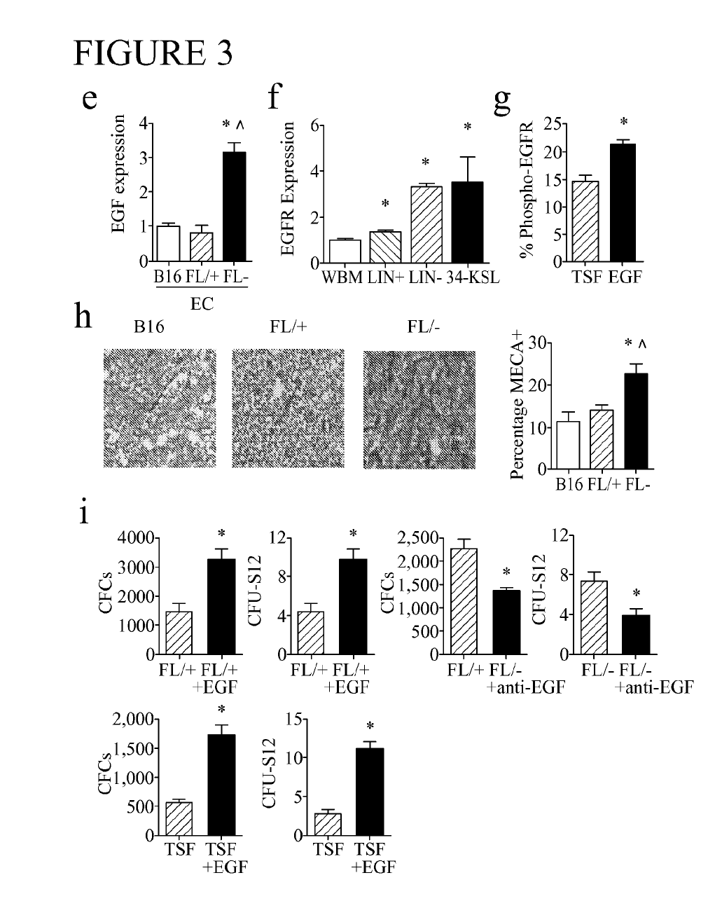

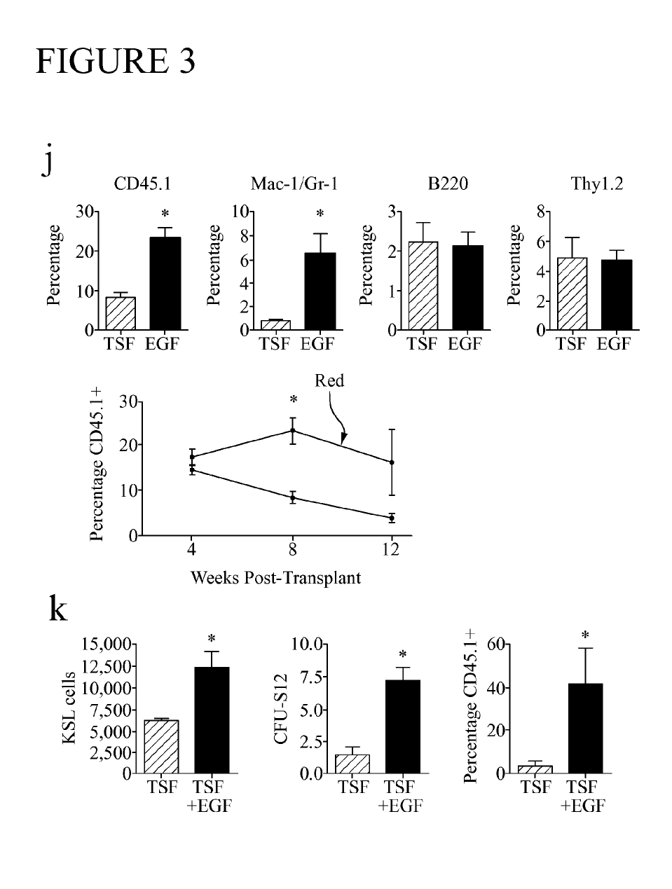

It was hypothesized that BM ECs regulate hematopoietic regeneration following injury and a genetic model was developed to delete BAK and BAX, which regulate the intrinsic pathway of apoptosis (Kirsch et al, Science 327:593-596 (2010)), in Tie2⁺ ECs as a means to protect BM ECs from radiation-induced injury. Following high dose TBI, Tie2Cre;Bak1^{-/-};Bax^{Fl/-} mice demonstrated significant protection of the BM vascular and HSC compartments as well as marked improvement in survival compared to Tie2Cre;Bak1^{-/-};Bax^{Fl/+} mice, which retain 1 allele of Bax, and wild type mice. In order to identify candidate secreted factors elaborated by Tie2⁺ BM ECs that might be responsible for the hematopoietic radioprotection in Tie2Cre;Bak1^{-/-};Bax^{Fl/-} mice, primary BM EC lines (CD45⁻ VWF⁺ Lectin⁺ AcLDL⁺) were generated from Tie2Cre;Bak1^{-/-};Bax^{FL/-} mice (FL/− ECs) and Tie2Cre;Bak1^{-/-};Bax^{FL+} mice (FL/+ECs), following previously described methods (Chute et al, Blood 105:576-583 (2005), Himburg et al, Nat. Med. 16:475-482 (2010), Chute et al, Blood 100:4433-4439 (2002), Yoder et al, Blood 109:1801-1809 (2007)). When BM KSL progenitor cells, which had been irradiated with 300 cGy in vitro, were plated in non-contact culture with FL/− ECs, a 3.2-fold increase was observed in total viable cells, a 6-fold increase in CFCs and a 3-fold increase in CFU-S12 recovered at day +7 compared to non-contact FL/+EC cultures (FIG. 3A). These results demonstrated that BM ECs from Tie2Cre;Bak1^{-/-};Bax^{FL/-} mice produced soluble factors that promoted the regeneration of hematopoietic stem/progenitor cells following radiation injury. In a complementary study, it was found that the addition of BM serum from irradiated Tie2Cre;Bak1^{-/-};Bax^{FL/-} mice to cultures of irradiated BM KSL cells induced a significant increase in the recovery of total cells and CFCs in 7 day culture. In contrast, irradiated BM KSL cells cultured identically with BM serum from Tie2Cre;Bak1^{-/-};Bax^{FL/+} mice yielded few viable hematopoietic cells (FIG. 3B). In order to identify candidate paracrine factors in the BM of Tie2Cre;Bak1^{-/-};Bax^{FL/+} mice which were responsible for the radioprotection of hematopoietic stem/progenitor cells, a cytokine array was performed on the BM serum from Tie2Cre;Bak1^{-/-};Bax^{FL/-} mice, Tie2Cre;Bak1^{-/-};Bax^{FL/+} mice and wild type C57B16 mice prior to and following 750 cGy TBI.

Several cytokines were identified that were significantly increased or decreased in concentration in BM serum from non-irradiated and irradiated Tie2Cre;Bak1^{-/-};Bax^{FL/-} mice versus Tie2Cre;Bak1^{-/-};Bax^{FL/+} mice (FIG. 3C). Several of these candidate proteins were screened, including IL17f, IL17, keratinocyte-derived chemokine (KC) and IL5, for in vitro regenerative or inhibitory activity on irradiated BM KSL cells. It was found that none of these proteins significantly altered the recovery of BM KSL cells in vitro following irradiation and none modulated the activity of FL/− ECs or FL/+ ECs in promoting BM KSL cell recovery after irradiation (data not shown). Epidermal growth factor (EGF), which was >18-fold increased in concentration in the BM serum of irradiated Tie2Cre;Bak1^{-/-};Bax^{FL/-} mice compared to irradiated Tie2Cre;Bak1^{-/-};Bax^{FL/+} mice, was also examined (FIG. 3D) and was expressed at 3-fold higher levels by FL/− ECs compared to FL/+ ECs (FIG. 3E). EGFR was expressed by wild type (C57B16) BM CD34⁻c-kit⁺sca-1⁺lineage⁻(34⁻KSL) cells, which are highly enriched for HSCs (Himburg et al, Nat. Med. 16:475-482 (2010)), and treatment of wild type BM KSL cells with EGF in vitro induced EGFR signaling as measured by EGFR phosphorylation (FIGS. 3F. 3G). In order to exclude the possibility that EGF enrichment in the BM of Tie2Cre;Bak1^{-/-};Bax^{FL/-} mice was due to autocrine secretion by BM HSCs, ELISA was performed on supernatants of BM KSL cells from Tie2Cre;Bak1^{-/-};Bax^{FL/-} mice and no detectable EGF was found (data not shown). Interestingly, immunohistochemical staining of femurs of Tie2Cre;Bak1^{-/-};Bax^{FL/-} mice, Tie2Cre;Bak1^{-/-};Bax^{FL/+} mice and C57B16 mice revealed an increased density of mouse endothelial cell antigen-positive (MECA⁺) vessels in Tie2Cre;Bak1^{-/-};Bax^{FL/-} mice compared to both control groups (FIG. 3H). Therefore, the increased concentrations of EGF in the BM serum of Tie2Cre;Bak1^{-/-};Bax^{FL/-} mice compared to Tie2Cre;Bak1^{-/-};Bax^{FL/+} mice and C57B16 mice may have been caused, in part, by increased density of EGF-secreting BM ECs in Tie2Cre;Bak1^{-/-};Bax^{FL/-} mice.

Gain-of-function studies were next performed to determine whether the addition of EGF to cultures of irradiated BM KSL cells with FL/+ ECs or cytokines alone (Thrombopoietin, Stem cell factor, Flt-3 ligand, TSF) could support HSC regeneration in vitro. The addition of 20 ng/mL EGF to non-contact FL/+ cultures caused a 2.2-fold increase in CFCs and a 2.6-fold increase in CFU-S12 recovered at day 7 compared to culture with FL/+ ECs alone (FIG. 3I). Conversely, when anti-EGF blocking antibody was added to cultures of irradiated BM KSL cells with FL/− ECs, a significant decrease in the recovery of both CFCs and CFU-S12 was observed (FIG. 3I). Most importantly, when EGF was added to irradiated BM KSL cells cultured with cytokines alone, a 3-fold increase in CFCs and a 4-fold increase in CFU-S12, compared to culture with cytokines alone, was observed (FIG. 3I). These data demonstrated that EGF acted directly on BM stem/progenitor cells to induce regeneration and did not depend upon EC-mediated effects. It was confirmed that EGF treatment promoted the regeneration of the HSC pool following radiation injury via competitive repopulation assays. At 8 and 12 weeks post-transplant, mice competitively transplanted with the progeny of irradiated, EGF-treated BM 34⁻KSL cells demonstrated 3- and 5-fold higher donor hematopoietic cell repopulation, respectively, compared to mice transplanted with the progeny of cytokine cultures alone (FIG. 3J).

In order to determine if activation of EGFR signaling could also promote the expansion of BM stem/progenitor cells in homeostasis, non-irradiated BM KSL cells were cultered in liquid suspension with cytokines with and without EGF in vitro. Remarkably, the addition of EGF to cytokine cultures of non-irradiated BM KSL cells caused a significant expansion of BM KSL cells and CFU-S 12 compared to the progeny of cytokine cultures alone (FIG. 3K). Moreover, recipient mice competitively transplanted with the progeny of BM 34⁻KSL cells cultured with cytokines+EGF displayed >10-fold increased donor hematopoietic cell repopulation at 12 weeks post-transplant compared to mice transplanted with the progeny of BM 34⁻KSL cells cultured with cytokines alone (FIG. 3K). These results demonstrate that EGF can also induce the expansion of. HSCs in steady state.

### Systemic Administration of EGF Induces HSC Regeneration In Vivo

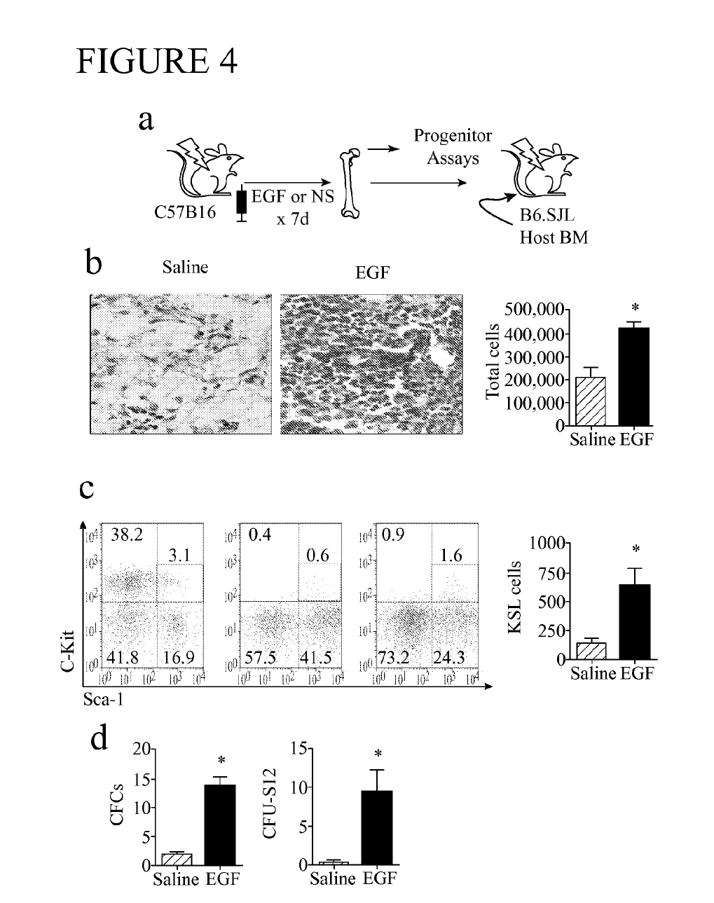

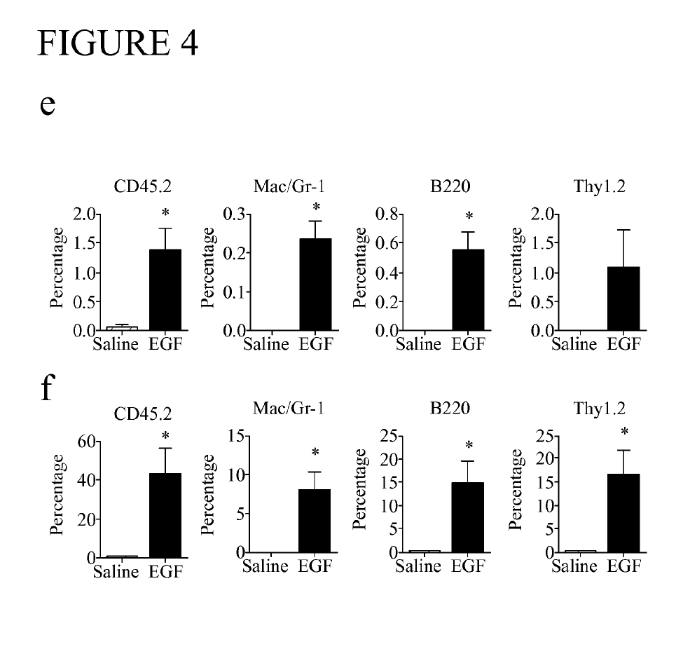

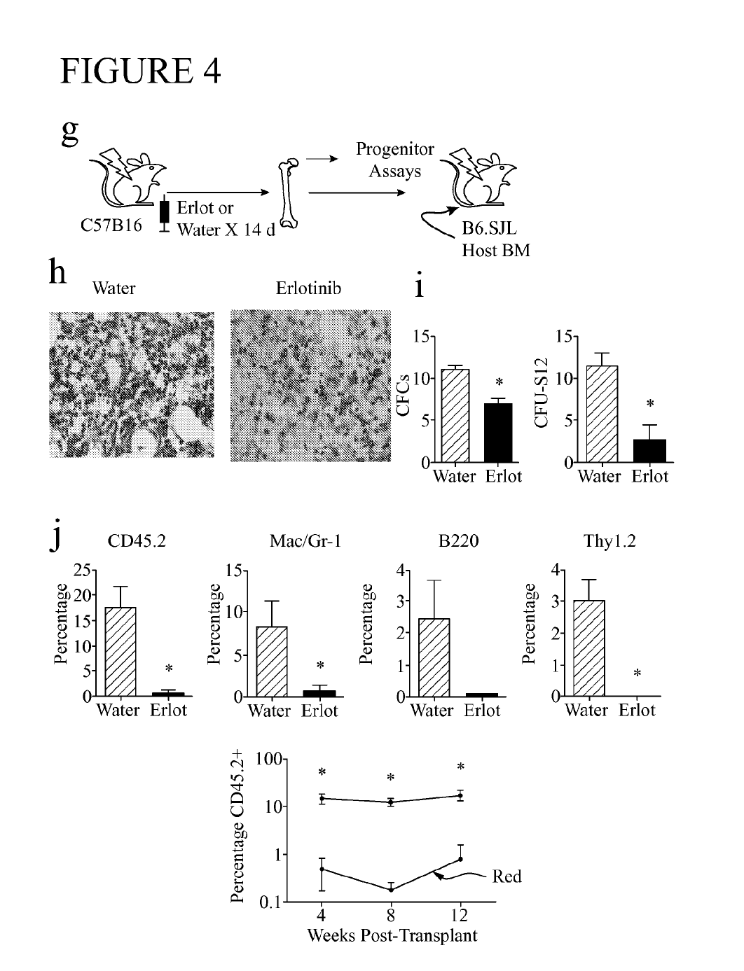

In order to determine if EGF signaling regulates HSC regeneration in vivo, hematopoietic reconstitution was measured in C57B16 mice following 700 cGy TBI and subsequent treatment with either EGF or saline beginning at +2 hrs post-TBI and then daily for 7 days (FIG. 4A). At day +7 following TBI, saline-treated mice demonstrated BM aplasia, whereas BM cellularity was largely preserved in EGF-treated animals (FIG. 4B). At the same time point, EGF-treated mice contained 2-fold increased BM cells, 6-fold increased BM KSL progenitor cells, 7-fold increased CFCs and 8-fold increased CFU-S12 compared to saline-treated mice (FIGS. 4B-4D). EGF-treated mice also contained significantly increased BM HSC content compared to saline-treated mice at day +7 following TBI, as measured by competitive repopulation assay (FIG. 4E). Mice transplanted with BM cells from irradiated, EGF-treated mice displayed increased multilineage reconstitution of myeloid cells, B cells and T cells at 12 weeks post-transplant compared to mice transplanted with BM cells from irradiated, saline-treated mice (FIG. 4E). Secondary transplant studies were also performed to assess whether EGF treatment augmented the regeneration of long term-HSCs (LT-HSCs) following 700 cGy TBI. Of note, secondary mice transplanted with BM cells from primary mice that received BM cells from donor mice at day +7 following TBI showed no significant engraftment at 12 weeks in either the EGF-treatment or saline-treatment groups (data not shown). However, secondary mice transplanted with BM cells from primary mice that received BM collected from irradiated, EGF-treated mice at day +14 following TBI displayed markedly increased donor cell repopulation compared to secondary mice transplanted identically in the saline-treatment control group (FIG. 4F). These data reveal that EGF treatment significantly augmented the regeneration of LT-HSCs in irradiated mice, but this effect of EGF on LT-HSC regeneration was only detectable in the model at 0.2 weeks following TBI.

### EGFR Inhibition Severely Impairs HSC Regeneration In Vivo

In order to determine if inhibition of EGFR signaling could alter HSC regeneration in vivo, mice were irradiated with 700 cGy TBI and treated with erlotinib, an EGFR antagonist, or water, via oral gavage beginning at day 0 and continuing daily through day +14 (FIG. 4G). At day +7 post-TBI, both erlotinib-treated and control mice demonstrated depletion of BM HSCs and progenitor cells (data not shown). At day +14, irradiated, control mice demonstrated recovery of BM cellularity while erlotinib-treated mice displayed persistent BM hypoplasia (FIG. 4H). Concurrent with this, irradiated control mice demonstrated recovery of BM CFCs and CFU-S12 at day +14, whereas erlotinib-treated mice displayed persistent, significant depletion of BM CFCs and CFU-S12 (FIG. 4I), Importantly, the most severe deficit in erlotinib-treated mice was in the HSC pool, which was essentially absent at day +14 post-TBI, as measured by competitive repopulation assay (FIG. 4J). Conversely, irradiated control mice displayed recovery of the HSC pool at day +14 following 700 cGy TBI (FIG. 4J). Erlotinib-treated mice displayed marked reduction in both short-term and longer-term HSCs compared to control mice as demonstrated by analysis of 4 week through 12 week donor cell engraftment in syngenic recipient mice (FIG. 4J). Taken together, these results demonstrated that pharmacologic inhibition of EGFR signaling severely impaired BM stem/progenitor cell regeneration following TBI.

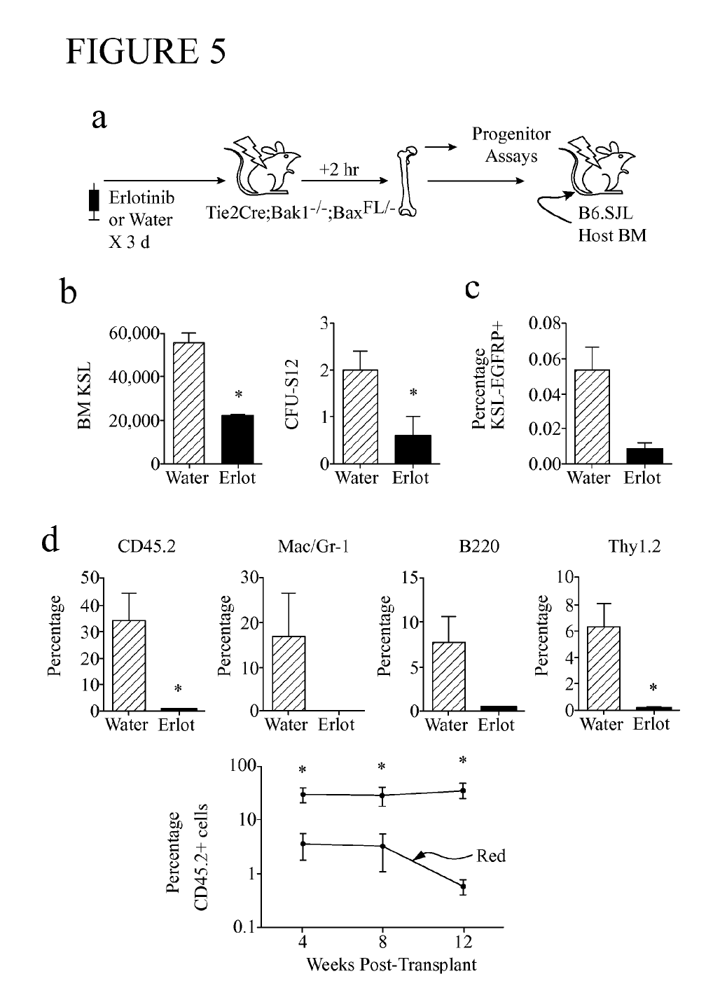

In order to determine whether EGF signaling was involved in mediating the radioprotection observed in Tie2Cre;Bak1^{-/-};Bax^{Fl/-} mice, a test was made of whether systemic administration of erlotinib would increase the radiosensitivity of the HSC pool in these mice (FIG. 5A). Erlotinib or water was administered to Tie2Cre;Bak1^{-/-};Bax^{Fl/-} mice beginning 3 days prior to 300 cGy TBI and BM HSC and progenitor cell content were evaluated at +2 hours following irradiation. Erlotinib-treated Tie2Cre;Bak1^{-/-};Bax^{Fl/-} mice demonstrated significantly decreased BM KSL cells and CFU-S12 following TBI compared to control, irradiated Tie2Cre;Bak1^{-/-};Bax^{Fl/-} mice (FIG. 5B). Importantly, this reduction in BM stem/progenitor cell content corresponded with a decrease in EGFR-phosphorylation in BM KSL cells in erlotinib-treated Tie2Cre;Bak1^{-/-};Bax^{Fl/-} mice (FIG. 5C). As expected, irradiated Tie2Cre;Bak1^{-/-};Bax^{Fl/-} mice demonstrated relative protection of the BM HSC pool following TBI. In contrast, erlotinib-treated, irradiated Tie2Cre;Bak1^{-/-};Bax^{Fl/-} mice displayed more than 20-fold decreased HSC content as measured via 4- to 12-week engraftment in competitively transplanted recipient mice (FIG. 5D). Mice transplanted with BM cells from erlotinib-treated Tie2Cre;Bak1^{-/-};Bax^{Fl/-} mice demonstrated reduced engraftment of myeloid cells, B cells and T cells at 12 weeks post-transplant compared to mice transplanted with BM cells from irradiated, control Tie2Cre;Bak1^{-/-};Bax^{Fl/-} mice (FIG. 5D). These results suggested that EGFR signaling was necessary for the radioprotection of the HSC pool observed in Tie2Cre;Bak1^{-/-};Bax^{Fl/-} mice.

### EGFR is Necessary for Hematopoietic Progenitor Cell Regeneration Following Irradiation

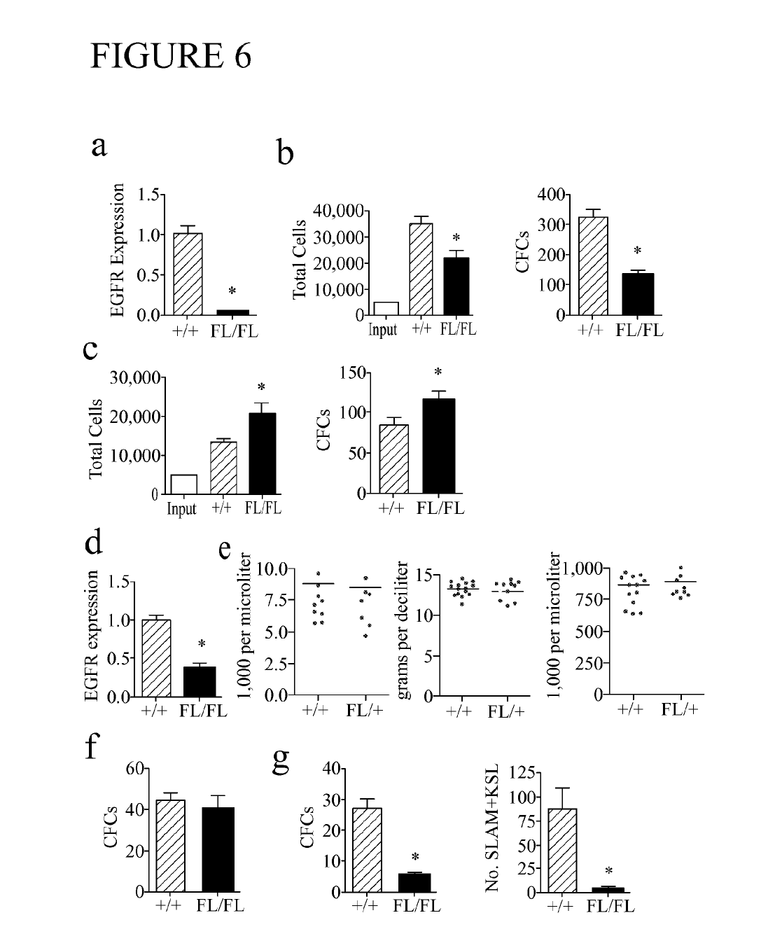

The pronounced delay in recovery of BM HSCs and progenitor cells in mice following TBI coupled with erlotinib treatment suggests an essential role for EGFR signaling in hematopoietic regeneration. However, erlotinib has been shown to inhibit kinases other than EGFR, including Jak2 and Src family kinases (Boehrer et al, Blood 111:2170-2180 (2008), Boehrer et al, Cell Cycle 10:3168-3175 (2011)). Therefore, a test was made of whether erlotinib acted specifically via EGFR inhibition in HSCs or via off-target effects to inhibit hematopoietic regeneration following TBI. VavCre;EGFR^{fl/fl} mice (EGFR^{fl/fl}) and VavCre;EGFR^{+/+} (EGFR^{+/+}) mice were generated and the deletion of EGFR expression in BM lineage-negative (lin⁻) cells was verified (FIG. 6A). BM lin⁻ cells from EGFR^{fl/fl} or EGFR^{+/+} mice were cultured in cytokine media with and without erlotinib for 72 hours (FIGS. 6B and 6C). EGFR^{fl/fl} BM lin⁻ cells demonstrated no significant effect of erlotinib treatment on total cell expansion or CFC production compared to cytokines alone (FIGS. 6B and 6C). In contrast, EGFR^{+/+} lin⁻ cells produced significantly less total cells and CFCs in erlotinib-treated cultures compared to EGFR^{+/+} lin⁻ cells cultured with cytokines alone and compared to EGFR^{-/-} lin⁻ cells cultured with erlotinib. These data demonstrate that erlotinib acts specifically via EGFR to inhibit BM progenitor cell proliferation.

In complementary studies, a comparison was made of the in vivo hematopoietic recovery of VavCre;EGFR^{+/+} mice and VavCre;EGFR^{fl/+} mice (EGFR^{fl/+}), which are heterozygous for expression of EGFR in hematopoietie cells, following myelosuppressive TBI (500 cGy). At baseline (pre-irradiation), EGFR^{fl/+} mice demonstrated decreased EGFR expression in BM lin⁻ cells relative to EGFR^{+/+} mice (FIG. 6D) and displayed no differences in complete blood counts or BM CFC content compared to EGFR^{+/+} mice (FIGS. 6E and 6F). However, at day +7 following 500 cGy TBI, EGFR^{fl/+} mice contained 5-fold decreased BM CFC content and 30-fold decreased BM SLAW⁺KSL cells, which are highly enriched for HSCs (Kiel et al, Cell 121:1109-1121 (2005)), compared to EGFR^{+/+} mice (FIG. 60). Taken together, these data suggest that EGFR signaling is necessary for normal BM stem/progenitor cell regeneration to occur following TBI.

### EGF Promotes HSC Survival and Increases HSC Cycling Following Radiation Injury

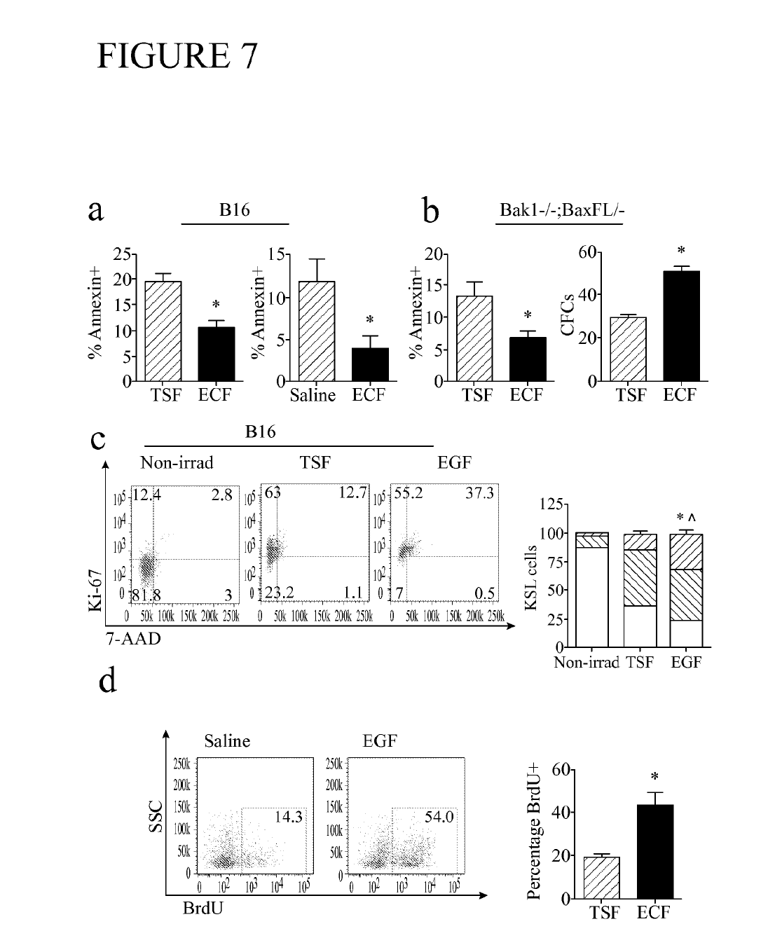

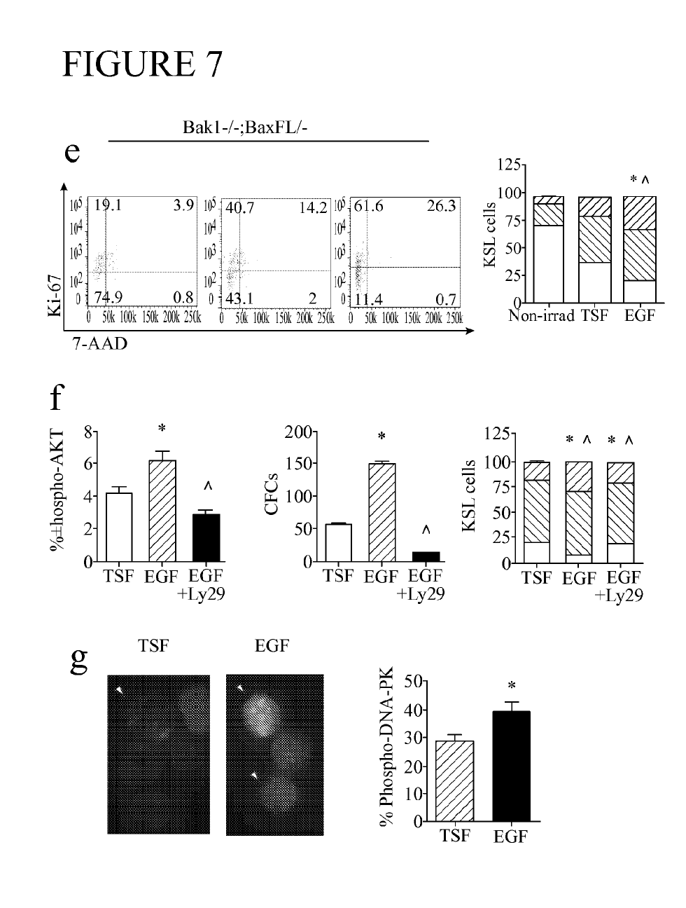

Activation of EGFR signaling can augment both cell survival and proliferation (Sordella et al, Science 305:1163-1167 (2004), Yang et al, Nature 480:118-122 (2011)). Thus, a test was next made of whether EGF treatment modulated HSC apoptosis or cell cycling following radiation exposure. BM KSL cells that were irradiated with 300 cGy in vitro and then treated with cytokines plus EGF contained 2-fold decreased. Annexin⁺KSL cells at 72 hours following irradiation compared to BM KSL cells treated with cytokines alone (FIG. 7A). Importantly, C57B16 mice that were irradiated with 700 cGy and then treated systemically with EGF×7 days contained 4-fold decreased Annexin⁺ BM hematopoietic cells compared to irradiated mice treated with saline×7 days (FIG. 7A). These results demonstrate that EGF treatment promotes HSC survival following radiation injury. A test was also made of whether EGF treatment could promote the survival of BM KSL cells from Tie2Cre;Bak1^{-/-};Bax^{Fl/-} mice after 300 cGy irradiation. Of note, irradiation of BM KSL cells from Tie2Cre;Bak1^{-/-};Bax^{Fl/-} mice produced less Annexin⁺ cells at 72 hours of cytokine culture compared to the identical dose of irradiation of BM KSL cells from C57B16 mice (FIGS. 7A and 7B). However, the addition of EGF further decreased the percentage of Annexin⁺ cells in culture of irradiated BM KSL cells from Tie2Cre;Bak1^{-/-};Bax^{Fl/-} mice compared to the same population treated with cytokines alone (FIG. 7B). This promotion of HSC survival in response to EGF corresponded with an increase in recovery of CFCs in culture compared to cytokine cultures alone (FIG. 7B). These results suggest that EGF is capable of promoting HSC survival following irradiation via mechanisms independent of BAK- and BAX-regulation of the intrinsic apoptotic pathway.

A comparison was made of the cell cycle status of HSCs that were treated in vitro with either cytokines alone or cytokines+EGF in vitro following 300 cGy irradiation. At baseline, the majority (>90%) of day 0, non-irradiated BM KSL cells resided in G₀/G₁ (FIG. 7C). At 72 hours of culture with cytokines alone, a mean of 27% of KSL cells remained in G₀, 54% had entered G₁ and 17% were in G₂/S/M phase (FIG. 7C). In contrast, in the EGF-treatment group, only 17% of KSL cells remained in G₀, 51% were in G₁ and 31% had entered G₂/S/M phase. Therefore, treatment with EGF caused a rapid and significant increase in the overall proliferation of BM stem/progenitor cells after irradiation and a near doubling of cells in G₂/S/M phase (FIG. 7C). In order to determine if EGF treatment also induced the proliferation of the HSC pool in vivo following TBI, BrdU incorporation in BM KSL cells from adult C57B16 mice was measured at day +7 following 700 cGy TBI and subsequent treatment with either EGF or saline daily×7 days. EGF-treated mice demonstrated a marked increase in BrdU incorporation in BM KSL cells at day +7 following TBI compared to saline-treated control mice (FIG. 7D). It was found that EGF treatment significantly increased the cycling of BM KSL cells from Tie2Cre;Bak1^{-/-};Bax^{Fl/-} mice following 300 cGy exposure in vitro, suggesting that EGF-mediated induction of HSC cycling following injury occurs independently of effects of Bak1 and Bax deletion (FIG. 7E).

EGFR signaling can activate multiple signaling cascades, including the MAPK and PI3k/Akt signaling pathways (Sordella et al, Science 305:1163-1167 (2004), Yang et al, Nature 480:118-122 (2011), Hynes and Lane, Nat. Rev. Cancer 5:341-354 (2005), Wheeler et al, Nat. Rev. Clin. Oncol. 7:493-507 (2010)). The activation of MAPK and Akt in BM KSL cells was interrogated following irradiation in the presence and absence of EGF treatment. EGF treatment of irradiated BM KSL cells in culture did not alter the phosphorylation of MAPK (data not shown) but did induce a 50% increase in Akt phosphorylation within 15 minutes (FIG. 7F). This induction of Akt in irradiated BM HSCs in response to EGF treatment corresponded with a 3-fold increase in BM CFC recovery in the EGF treatment group at 72 hours following radiation exposure (FIG. 7F). Furthermore, treatment of irradiated BM KSL cells with Ly294002, a PI3K inhibitor which prevents Akt phosphorylation, blocked EGF-mediated Akt phosphorylation and prevented the recovery of BM progenitor cells in response to EGF (FIG. 7F). These results demonstrate that EGF induces Akt signaling in BM HSCs following radiation injury and EGF-mediated regeneration of BM progenitor cells is dependent upon Akt activation. A further examination was made of whether inhibition of Akt signaling could negate EGF-mediated induction of BM stem/progenitor cell cycling following irradiation. Indeed, irradiated BM KSL cells that were treated with EGF+Ly294002 demonstrated significantly decreased entry into G₂/S/M phase and increased percentage of G₀ cells compared to KSL cells treated with EGF alone (FIG. 7F).

In addition to activation of the PI3k/Akt pathway, EGFR signaling can lessen radiation-induced DNA damage via rapid induction of the DNA repair enzyme, DNA-PK, which mediates non-homologous end-joining (NHEJ) repair (Liccardi et al, Cancer Res. 71:103-1114 (2011), Kriegs et al, DNA Repair (Amst) 9:889-897 (2010)). BM lin⁻ cells that were irradiated with 300 cGy and then treated in culture with and without EGF were interrogated for evidence of upregulation of activated DNA-PK. Immunohistochemical staining for phospho-DNA-PK, the activated form of DNA-PK, revealed a significant increase in phospho-DNA-PK levels in BM lin⁻ cells within 15 minutes of treatment with EGF as compared to irradiated BM lin⁻ cells treated with cytokines alone (FIG. 7G). FACS analysis of phospho-DNA-PK levels also revealed a significant increase in phospho-DNA-PK levels in BM lin⁻ cells at 1 hour of treatment with EGF compared to cytokines alone (FIG. 7G). These data demonstrate that EGF rapidly induces the DNA repair machinery in irradiated HSCs and suggest an additional mechanism through which EGFR activation can promote HSC survival following radiation injury.

### Systemic Administration of EGF Improves the Survival of Lethally Irradiated Mice

Since pharmacologic and genetic modulation of EGFR signaling in mice significantly altered hematopoietic regeneration following TBI, a determination was made of whether pharmacologic modulation of EGFR signaling could affect the survival of mice following lethal doses of TBI. Adult C57B16 mice were treated with 10 μg/G erlotinib or water (via oral gavage) beginning 3 days prior to 700 cGy TBI and continuing for 14 days post-irradiation. Fifty-three percent (8 of 15) of control, irradiated mice remained alive and well through day +30. In contrast, none (0 of 15) of the erlotinib-treated mice survived beyond day +27

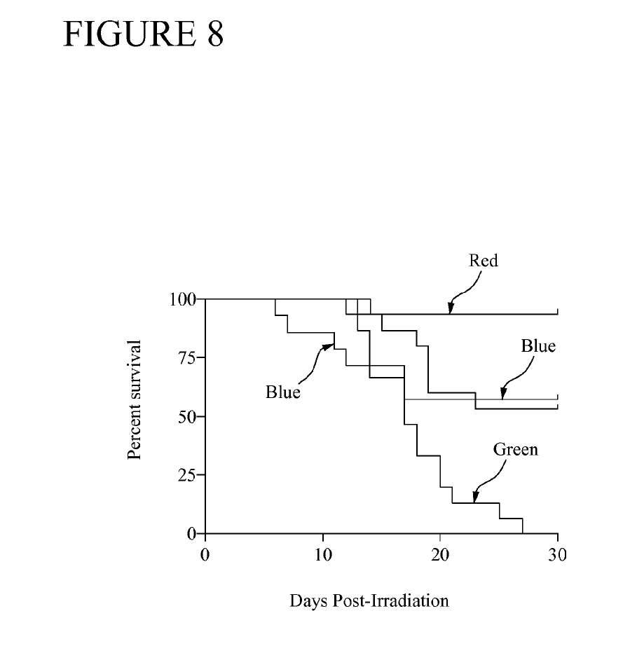

(FIG. 8). An additional cohort of age-matched C57B16 mice was irradiated with 700 cGy and treated intraperitoneally with either 0.5 μg/G EGF or saline×7 days, beginning 2 hours post-TBI. Comparable to mice treated with water gavage, 57% of saline-treated mice (8 of 14) survived through day +30 (FIG. 8). However, 93% (14 of 15) of EGF-treated mice remained well through day +30. These results demonstrate that systemic administration of EGF can substantially improve survival following lethal dose TBI and that EGFR signaling has an essential role in regulating survival after TBI.

In summary, recent studies have suggested that hematopoietic regeneration in vivo is regulated by BM ECs (Salter et al, Blood 113:2104-2107 (2009), Butler et al, Cell Stem Cell 6:251-264 (2010), Hooper et al, Cell Stern Cell 4:263-274 (2009)). However, the mechanisms through which BM ECs regulate hematopoietic regeneration remain largely unknown. Identification of the mechanisms which govern hematopoietic regeneration could have broad implications for the development of therapies to accelerate hematologic recovery in patients receiving myelosuppressive chemo- or radiotherapy or undergoing stem cell transplantation (Appelbaum, N. Engl. J. Med. 357:1472-1475 (2007)). The study described above demonstrates that EGF, identified via a screen of BM serum from radioprotected mice bearing deletion of BAK and BAX in Tie2⁺ ECs, potently mitigates radiation injury to the HSC compartment. Treatment with EGF significantly increased recovery of BM HSCs and progenitor cells in vitro following radiation exposure compared to cytokines alone. Furthermore, systemic administration of EGF potently increased both hematopoietic regeneration and the overall survival of mice compared to irradiated, control mice. Conversely, treatment with the EGFR inhibitor, erlotinib, markedly delayed the recovery of BM stem and progenitor cells and significantly decreased the survival of irradiated mice compared to irradiated, control mice. Taken together, these studies demonstrate that EGFR signaling regulates the response of the HSC pool and the hematopoietic system as a whole to ionizing radiation. Of note, since EGF has mitogenic and reparative effects on several non-hematopoietic tissues that are affected by radiation injury (e.g. gut, lung), it is possible that EGF action on these non-hematopoietic tissues could contribute to EGF-mediated improvement in survival following TBI. Nonetheless, the results suggest that systemic administration of EGF has therapeutic potential to accelerate hematopoietic recovery in stem cell transplant patients who have received TBI conditioning, as well as for the victims of acute radiation sickness, a condition for which few proven treatments exist (Chen et al, PLoS One 5:e11056 (2010), Li et al, J. Radiat. Res. (Tokyo) 52:712-716 (2011))).

Since erlotinib has recently been shown to mediate cellular effects via inhibition of enzymes other than EGFR (e.g. Jak2 and SRC family kinases) (Boehrer et al, Blood 111:2170-2180 (2008), Boehrer et al, Cell Cycle 10:3168-3175 (2011)), a genetic model of VavCre;EGFR^{fl/fl} mice was used to determine the specific role of EGFR in regulating the hematopoietic response to radiation, In vitro studies demonstrated that erlotinib treatment had no effect on EGFR^{-/-} BM HSCs in culture, whereas erlotinib treatment of EGFR^{+/+} BM HSCs significantly inhibited both cell expansion and CFC production in culture. Therefore, erlotinib acted specifically through EGFR inhibition to diminish hematopoietic progenitor cell recovery in this model. Importantly, VavCre;EGFR^{fl/+} mice, which are heterozygous for EGFR expression, displayed significantly decreased BM CFC content and SLAM⁺KSL HSCs¹ at day +7 following 500 cGy TBI compared to VavCre;EGFR^{+/+} mice, which retained both EGFR alleles. These results demonstrate that EGFR signaling is necessary for normal hematopoietic regeneration to occur following TBI.

These studies indicate that EGFR signaling regulates two central mechanisms through which HSCs responds to stress: apoptotic cell death and cell cycling. Since erlotinib treatment significantly decreased the radioprotection of the BM HSC pool that was otherwise observed in Tie2Cre;Bak1^{-/-};Bax^{Fl/-} mice, this suggested that EGFR signaling perhaps also regulated the HSC response to radiation injury independently of BAK- and BAX-mediated apoptosis. Therefore, the effect of EGF treatment was directly tested on irradiated BM HSCs from Tie2Cre;Bak1^{-/-};Bax^{Fl/-} mice in culture. Interestingly, while BM HSCs from Tie2Cre;Bak1^{-/-};Bax^{Fl/-} mice underwent less cell death in response to ionizing radiation compared to BM HSCs from wild type mice, EGF treatment further decreased radiation-induced death of HSCs from Tie2Cre;Bak1^{-/-};Bax^{Fl/-} mice compared to treatment with cytokines alone. These data demonstrate that BAK and BAX clearly regulate HSC apoptosis in response to ionizing radiation, but EGF can modulate HSC cell death via mechanisms independent of BAK and BAX. One such potential mechanism which is supported by the results is that of EGFR-mediated induction of DNA-PK, which regulates non-homologous end-joining (NHEJ) repair of radiation-induced DNA damage. Studies of primary cancers and cancer cell lines have shown that EGFR activation promotes NHEJ repair of radiation-induced double strand DNA breaks via induction of DNA-PK (Kriegs et al, DNA Repair (Amst) 9:889-897 (2010), Mukherjee et al, Semin. Radiat. Oncol. 20:250-257 (2010), Szumiel, Cell Signal 18:1537-1548 (2006), Das et al, Cancer Res. 67:5267-5274 (2007)). Golding et al (Cancer Biol. Ther. 8:730-738 (2009)) also demonstrated that EGFR-induced NHEJ repair of radiation-induced DNA damage in glioma cells was Akt-dependent. This result is consistent with the observation that EGF-mediated hematopoietic progenitor cell regeneration could be abolished by Akt inhibition and suggests that Akt could be regulating DNA repair in irradiated HSCs in response to EGF (Chan et al, Proc. Natl. Acad. Sri. USA 106:22369-22374 (2009), Bussink et al, Lancet Oncol. 9:288-296 (2008), Hay, Cancer Cell 8:179-183 (2005)). Importantly, treatment with erlotinib prior to irradiation has been shown to block EGFR-mediated activation of DNA-PK, thereby increasing the radiosensitivity of cancer cells (Kriegs et al, DNA Repair (Amst) 9:889-897 (2010), Szumiel, CellSignal 18:1537-1548 (2006)). This radiosensitizing effect of erlotinib could explain, at least in part, the observation that systemic administration of erlotinib prior to TBI in Tie2Cre;Bak1^{-/-};Bax^{Fl/-} mice caused a substantial depletion of the HSC pool compared to irradiated control Tie2Cre;Bak1^{-/-};Bax^{Fl/-} mice. Lastly, in addition to the direct effects of EGFR signaling on DNA repair, it has also been shown that EGF treatment strongly induces BM stem/progenitor cell cycling following irradiation in an Akt-dependent manner. This Akt-dependent induction of HSC proliferation represents another mechanism through which EGF promotes hematopoietic regeneration independently of BAK and BAX.

Here, a previously unknown role of EGF and EGFR signaling in regulating HSC regeneration and survival following radiation-induced myelosuppression has been delinated. EGFR is widely expressed in epithelial and neuroectodermal tissues and EGF induces proliferation and anti-apoptotic effects in epithelial cells and endothelial cells, promotes wound healing and can be tumorigenic in epithelial cells (Prigent and Gullick, EMBO J. 13:2831-2841 (1994), Dittman et al, J. Biol. Chem. 280:31182-31189 (2005), Rodemann et al, Int. J. Radiat. Biol. 83:781-791 (2007), Wang et al, Invest. Ophthalmol. Vis. Sci. 51:2943-2948 (2010), Cardo-Vila et al, Proc. Natl. Acad. Sci. USA 107:5118-5123 (2010), Ji et al, Cancer Cell 9:485-495 (2006)). However, prior studies suggested that EGFR was not expressed on hematopoietic stem cells (Pain et al, Cell 65:37-46 (1991), von Ruden and Wagner, EMBO J. 7:2749-2756 (1988), Real et al, Cancer Res. 46:4726-2731 (1986)). While EGFR was recently shown to be expressed at low levels on BM ckit⁺lin⁻ progenitor cells ((Chan et al, Proc. Natl. Acad. Sci. USA 106:22369-22374 (2009), Ryan et al, Nat. Med. 16:1141-1146 (2010)), EGF has not been previously shown to directly regulate HSC self-renewal or regeneration. One prior study suggested that the addition of EGF to stromal cell co-cultures inhibited hematopoietic progenitor cell growth in vitro, although these effects were mediated via indirect effects on stromal cells (Dooley et al, J. Cell Physiol. 165:386-397 (1995)). In contrast, EGF has a demonstrated function in regulating stem cell activities in non-hematopoietic tissues. It was recently shown that EGFR signaling regulated the maintenance and differentiation of neuronal stern cells (Aguirre et al, Nature 467:323-327 (2010)) and EGF-responsive, human neuronal stem cells have been described (Shih et al, Blood 98:2412-2422 (2001)). In addition, EGFR has been shown to be required for efficient liver regeneration following hepatectomy (Natarajan et al, Proc. Natl. Acad. Sci. USA 104:17081-17086 (2007)). These studies suggest the potential for a more general role of EGFR signaling in regulating stem cell function in non-hematopoietic tissues.

Recently, Ryan et al (Nat. Med. 16:1141-1146 (2010)) reported that inhibition of EGFR signaling facilitated GCSF-mediated mobilization of hematopoietic progenitor cells in mice. In this study, no direct effects of EGF or EGFR antagonists on BM progenitor cell mobilization were demonstrated in the absence of GCSF treatment and no effects on HSC content, proliferation or function were described (Ryan et al, Nat. Med. 16:1141-1146 (2010)). Here it is shown that EGF acts directly on HSCs to induce HSC expansion, promote hematopoietic regeneration and improve the survival of mice following TBI. It is also shown that EGF mediates proliferative and regenerative effects on irradiated HSCs via induction of Akt signaling. These observed effects of EGF on HSC growth are comparable those described for fibroblast growth factor 1 (FGF1), which also activates PI3k/Akt signaling, suggesting a possible convergence of action of EGF and FGF1 on critical signaling pathways in HSCs (Zhang and Lodish, Blood 105:4314-4320 (2005), Hashimoto et al, J. Biol. Chem. 277:32985-32991 (2002)). In contrast to EGF, systemic administration of erlotinib profoundly delays hematopoietic regeneration and significantly worsens the survival of mice following TBI. Complementary genetic studies suggest an essential role for EGFR in regulating hematopoietic regeneration in vivo. Taken together, these results demonstrate that pharmacologic administration of EGF or other EGFR ligands has therapeutic potential to accelerate hematopoietic reconstitution in patients following radiation injury or TBI-based conditioning for stem cell transplantation. The recent radiation crisis in Fukushima, Japan underscores the importance of this mechanism for the potential treatment of acute radiation sickness, which can cause life threatening BM failure and for which few treatments exist.

All documents and other information sources cited herein are hereby incorporated in their entirety by reference.

1. A method of inducing or accelerating hematologic recovery comprising administering to a subject in need thereof epidermal growth factor (EGF) in an amount sufficient to effect said induction or acceleration.

2. A method of promoting hematopoietic stem cell survival or proliferation during or following radiation injury comprising administering to a subject in need thereof EGF in an amount sufficient to effect said promotion or proliferation.

3. The method according to claim 1 wherein said subject is a mammal.

4. The method according to claim 3 wherein said mammal is a human.

5. The method according to claim 1 wherein said subject has undergone or is undergoing bone marrow transplantation.

6. The method according to claim 1 wherein said subject has undergone or is undergoing a myelotoxic therapy

7. The method according to claim 6 wherein said myelotoxic therapy is radiation therapy or chemotherapy.

8. The method according to claim 1 wherein said EGF is administered systemically.

9. The method according to claim 1 wherein said administration of EGF causes an acceleration in recovery of hematopoietic stem and progenitor cells.

10. The method according to claim 1 wherein said EGF is recombinant EGF.

11. The method according to claim 1 wherein said EGF is human EGF.

12. A method of expanding hematopoietic stem and progenitor cells comprising contacting said cells with an amount of EGF sufficient to effect said expansion.

13. The method according to claim 12 wherein said cells are in vitro.

14. The method according to main 12 wherein said cells are in vivo.

15. Use of EGF in the manufacture of a medicament for inducing or accelerating hematologic recovery in a subject in need thereof.

16. Use of EGF in the manufacture of a medicament for promoting hematopoietic stem cell survival or proliferation during or following radiation injury in a subject in need thereof.
